(19) United States
(12) Patent Application Publication
(10) Pub. No.: US 2012/0265189 A1
(43) Pub. Date: Oct. 18, 2012

(54) METHOD OF CAUTERIZATION WITH A CRYOPROBE

(75) Inventors: Timothy J. Davis, Coon Rapids, MN (US); Satish Ramadhyani, Minneapolis, MN (US); William M. Jacqmein, Woodbury, MN (US); Vineel Vallapureddy, Coon Rapids, MN (US); Luan Thien Chan, Coon Rapids, MN (US)

(73) Assignee: GALIL MEDICAL INC., Arden Hills, MN (US)

(21) Appl. No.: 13/087,079

(22) Filed: Apr. 14, 2011

(51) Int. Cl. A61B 18/02

(52) U.S. Cl. 606/22

(57) ABSTRACT

The invention is a method for using a cryoprobe for cryoablating, thawing and/or cauterizing a tissue during a cryosurgical procedure. An embodiment of the cryosurgical system comprises a controller including a cryoprobe with an electrical heating element. The controller operates the electrical heating element for heating a treatment head of the cryoprobe to a temperature sufficient for cauterizing the tissue in the vicinity of the treatment head. Additionally, the controller regulates a supply of a heating gas and a cooling gas, respectively, for thawing and cryoablating the tissue in the vicinity of the treatment head. The controller is configured for operating a plurality of cryoprobes connected thereto.

### TECHNICAL FIELD

The present invention relates to a method of cauterizing a tissue using a cryoprobe during a cryosurgical procedure.

### BACKGROUND

Cryosurgical systems according to the prior art comprise one or more cryoprobes connected to a cryogas supply module which includes one or more cryogas sources and a controller. The controller is typically designed to receive control commands from a surgeon and, following those commands, to control valves governing delivery of cryogas from the cryogas sources to the connected cryoprobes. In this manner a surgeon, by commanding actions of the controller, controls delivery of cryogas to the cryoprobes, thereby controlling cooling and heating of those cryoprobes and the tissue in their vicinity.

Cryoprobes are well known devices used for therapeutic freezing and thawing of target tissue such as tumor. One class of cryoprobes utilizes the Joule-Thomson effect to produce cooling or heating. In these probes, a gas is passed from a first region of the device, where it is held under higher pressure, to a second region of the device, wherein it is enabled to expand to a lower pressure. This expansion, and the associated Joule-Thomson effect may occur in a simple conduit such as a capillary tube, or it may occur in an orifice, generally referred to as a Joule-Thomson orifice, through which gas passes from a first, higher pressure, region of the device to a second, lower pressure, region of the device. In some embodiments, a cryoprobe further includes a heat exchanger that is used to pre-cool gases within a first region of the device, prior to their expansion into a second region of the device. Effective operation of the cryoprobes depends on the availability of the cryogases at their specified pressures. Too low a pressure may result in inadequate cooling or heating performance.

Generally, the cryogas sources are separate gas tanks containing a high-pressure cooling gas and a high-pressure heating gas. The term “cooling gas,” as is well known in the art, refers to a gas which, at room temperature or colder, has the property of becoming colder when it is permitted to expand from a region of higher pressure into a region of lower pressure and may to some extent liquefy creating a pool of liquefied gas. Examples of “cooling gases” include argon, nitrogen, air, krypton, CO₂, CF₄, xenon, and various other gases. In a cryoprobe, the cooling gas is typically permitted to expand within the tip at the distal end of the cryoprobe whereat the expansion of the gas results in temperatures at or below those necessary for cryoablating a tissue in the vicinity of the tip of the cryoprobe. Typically, argon is used as the cooling gas for cooling the cryoprobes to sufficiently low temperatures for cryoablating the tissue in the vicinity of the tips of the cryoprobes.

The term “heating gas,” as is well known in the art, refers to a gas which, at room temperature or warmer, has the property of becoming hotter when it is permitted to expand from a region of higher pressure into a region of lower pressure. Helium is an example of a “heating gases.” In a cryoprobe, the heating gas is typically permitted to expand within the tip at the distal end of the cryoprobe whereat the expansion of the gas results in temperatures at or above those necessary for thawing a cryoablated tissue. Typically, helium is used as the heating gas for heating the cryoprobes to thaw the tissue in the vicinity of the tips of the cryoprobes for the purpose of un-freezing the cryoprobes from the cryoablated tissue.

During the course of cryosurgical procedures it is often necessary to cauterize or coagulate tissue to control bleeding. While a heating gas, such as helium, can be used advantageously to raise the temperature of the cryoprobe to a level sufficient for inducing thawing, they do not generate sufficient energy upon expansion to heat a cryoprobe to temperatures necessary for cauterizing tissue.

Electrosurgical devices are known which utilize electrical current for tissue cauterization. U.S. Pat. Nos. 1,983,669 and 4,637,392 disclose electrical cauterization devices in which electrodes are disposed about the surface of a probe. As current passes through the tissue, some energy is absorbed into the tissue causing tissue temperature to rise. The rising temperature of the tissue denatures tissue protein molecules and facilitates coagulation. Among the drawbacks of such devices is the potential that the electrodes will become overheated, and the denatured proteins will weld to the electrode on the outer surface of the probe. This can result in tissue searing or dessication, or in tissue being torn from the surgical site as the probe is removed from the patient. Such a tear can result in bleeding or the reopening of a wound. A further problem results from tissue collecting over the probe and the need to remove tissue from the electrode before continuing to use the device. Tissue stuck to the probe interferes with the delivery of energy to the surgical site. This interference limits the depth of penetration of energy into the tissue and thereby limits the depth of cauterization. Because of these drawbacks these devices are impractical for certain surgical procedures. Moreover, it can be inconvenient to use such cauterization devices during certain surgical procedures because cryoablation and cauterization must be performed with separate instruments.

Electro-surgical processes using radio frequency (RF) has also been used for cauterizing tissue. The RF units generate heat by using high frequency electrical current and the resistive nature of tissue to produce heat. This technique requires a bulky generator and heavy electrical components to operate. Typically, RF electrocautery units require a power lead cable to the electro-surgical hand instrument and a large surface area grounding pad. More often than not, radio frequency surgical units are bulky expensive units which require a cable connection. Employing RF cauterization in a surgical operation may add significant cost to the procedure because the grounding pad, cable and handpiece must all be either re-sterilized or replaced in the case of disposable use.

Accordingly, there exists a need for a cryosurgical system that can provide all three functions, viz., cryoablation, thawing and cauterization, with a single cryosurgical instrument.

### SUMMARY

Embodiments of the invention comprise a cryosurgical system and a method for using the cryosurgical system configured for cryoablating a tissue, thawing the cryoablated tissue and cauterizing the tissue. The cryosurgical system includes a cryoprobe comprising both a Joule-Thomson orifice and an electrical heating element at a distal end of the cryoprobe. The cryosurgical system also includes a controller both for regulating a flow of cryogas from a cryogas source to the cryoprobe and for regulating electrical power supplied to the electrical heating element. As such, the controller is configured for maintaining a temperature proximate the distal end within a predetermined range for cryoablating the tissue, for thawing the tissue and for cauterizing the tissue.

### BRIEF DESCRIPTION OF THE DRAWINGS

FIG. 1A is a schematic of a cryosurgical system comprising a cryoprobe having an embedded electronic module in accordance with an embodiment of the invention;

FIG. 1B is a block diagram of an embodiment of the electronic module of FIG. 1A;

FIG. 2 is a schematic of a cryosurgical system in accordance with an alternate embodiment of the invention;

FIG. 3 is a schematic of a cryosurgical system in accordance with another embodiment of the invention;

FIG. 4 is a flowchart of an embodiment of a method for cauterizing a tissue during a cryosurgical procedure using a cryoprobe of the present invention;

FIG. 5 is a flowchart of an embodiment of a method for heating a cryoprobe of the present invention to temperatures sufficient for cauterizing a tissue;

FIG. 6 is a flowchart of another embodiment of a method for heating a cryoprobe of the present invention to temperatures sufficient for cauterizing a tissue; and

FIG. 7 is a flowchart of an alternate embodiment of a method for heating a cryoprobe of the present invention to temperatures sufficient for cauterizing a tissue.

### DETAILED DESCRIPTION

While multiple embodiments of the instant invention are disclosed, alternate embodiments may become apparent to those skilled in the art. The following detailed description describes only illustrative embodiments of the invention with reference to the accompanying drawings. It should be clearly understood that there is no intent, implied or otherwise, to limit the invention in any form or manner to that described herein. As such, all alternative embodiments are considered as falling within the spirit, scope and intent of the instant invention.

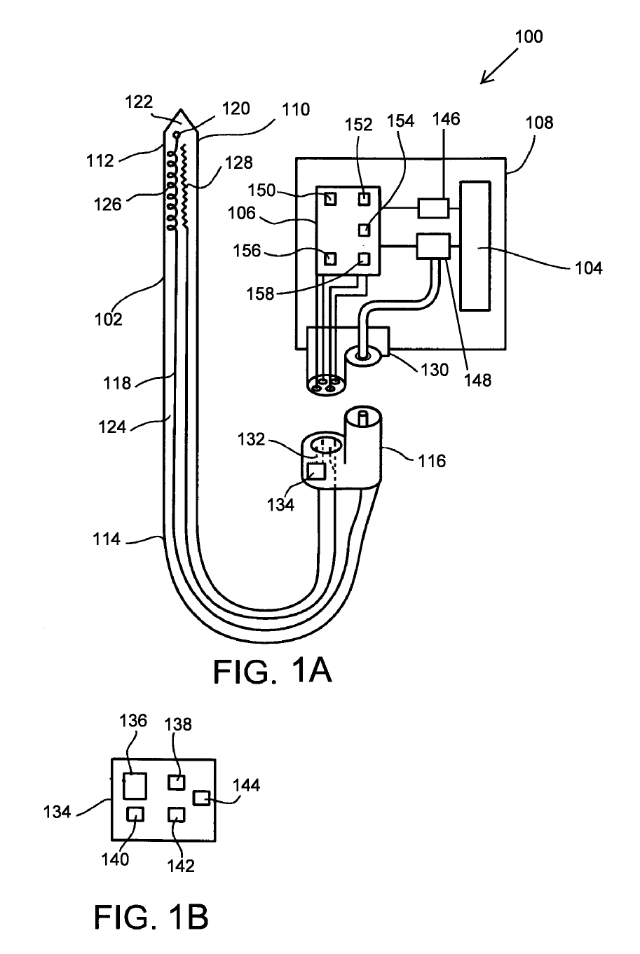

FIG. 1A illustrates cryosurgical system 100 comprising cryoprobe 102, cryogas source 104 for supplying a cryogas to cryoprobe 102, and controller 106 for controlling delivery of cryogas from cryogas source 104 to cryoprobe 102. In this non-limiting exemplary embodiment cryogas source 104 and controller 106 are shown housed in common cabinet 108. In alternate embodiments, cryogas source 104 is located external to and in fluid communication with cabinet 108. Cryoprobe 102 comprises distal portion 110 including treatment head 112 coolable by delivery thereto of a cryogas, flexible hose portion 114 and connector 116 on a proximal portion of cryoprobe 102. Cryogas supply conduit 118 supplies a cryogas (high-pressure cooling gas such as argon, or high-pressure heating gas such as helium) to Joule-Thomson orifice 120 in expansion chamber 122 in treatment head 112. Cryogas exhaust conduit 124 carries the expanded gas away from head 112 and back to connector 116. Heat exchanger 126 positioned in or near head 112 provides pre-cooling of high-pressure gas approaching treatment head 112. However, it is to be understood that although Joule-Thomson cooling is presented in this exemplary embodiment, cooling by evaporation of a liquefied cryogas, or any other form of cooling, falls within the scope of the present invention. Although not explicitly shown, alternate embodiments of cryoprobe 102 include a non-stick coating on distal portion 110 to prevent or minimize the tissue being treated from sticking or welding to treatment head 112. Exemplary bio-compatible coating material, as are well known in the art, include Teflon, fluorinated polymers, non-polar material, hydrophobic material, among others.

As is well known to one skilled in the art, cryoablation often causes treatment head 112 to freeze/stick to the tissue being treated because of the relatively low temperatures resulting from the expansion of the cooling gas. Accordingly, it is necessary to heat the frozen tissue to a temperature sufficiently high for disengaging treatment head 112 from the tissue. As is also well known to one skilled in the art, a heating gas, such as helium, is generally used for this purpose.

During the course of cryosurgical procedures it is often necessary to cauterize or coagulate tissue to control bleeding. While a heating gas can be used advantageously to raise the temperature of treatment head 112 to a level sufficient for thawing the tissue, such heating gases do not generate sufficient energy upon expansion to heat a cryoprobe to temperatures necessary for cauterizing tissue.

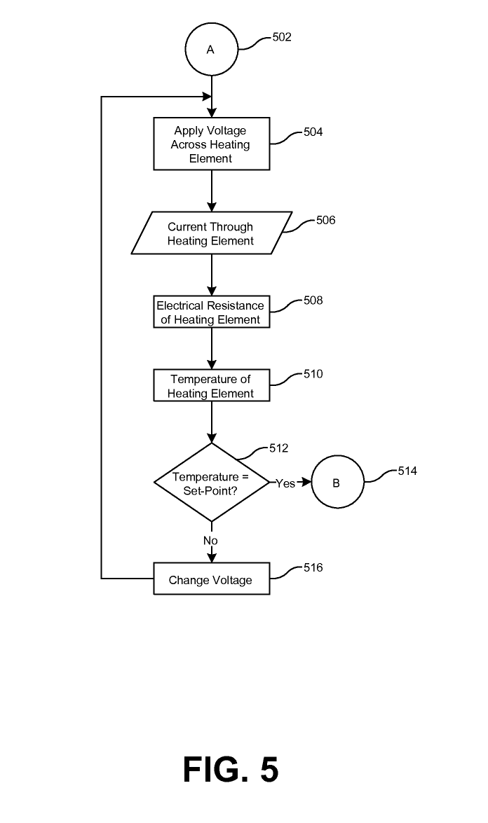

Cryoprobe 102, in accordance with an embodiment of the present invention, includes electrical heating element 128 for heating treatment head 112 to temperatures sufficiently high for cauterizing the tissue. In FIG. 1, electrical heating element 128 is shown integrated with heat-exchanger 126 to provide heating of head 112. In other embodiments, electrical heating element 128 is positioned elsewhere in cryoprobe 102 to provide heating of head 112. In accordance with an embodiment of the invention, electrical heating element 128 is of a low thermal mass for enabling both rapid heat-up when electrical power is applied and rapid cool-down when electrical power is terminated. Electrical heating element 128, in some embodiments of the invention, exhibits a negative temperature coefficient characteristic in that the electrical resistance of electrical heating element 128 decreases as the temperature of electrical heating element 128 increases. In alternate embodiments of the invention, electrical heating element 128 exhibits a positive temperature coefficient characteristic in that the electrical resistance of electrical heating element 128 increases as the temperature of electrical heating element 128 increases. An embodiment of controller 106 is configured for regulating the temperature of electrical heating element 128, and therefore also the temperature of treatment head 112 to a specified value. This is accomplished using methods well known in the art as described herein below with reference to FIGS. 5, 6 and 7. An alternate embodiment of controller 106 is configured for regulating the electrical power supplied to electrical heating element 128 using methods well known in the art such as regulating only the electrical voltage applied across heating element 128 or regulating only the flow of electrical current flowing through heating element 128 or regulating both the electrical voltage applied across heating element 128 and the flow of electrical current flowing through heating element 128.

Connector 116 on a proximal portion of cryoprobe 102 is used for connecting cryoprobe 102 to socket 130 on cabinet 108 for providing gas connection to cryogas source 104 and electrical/electronic connection to controller 106. In this exemplary embodiment, connector 116 is shown comprising power and data links 132, which may be a combined power and data link, and electronic module 134 embedded within connector 116. Power and data links 132 supply electrical power to cryoprobe 102, such as to electrical heating element 128 and electronic module 134. Furthermore, power and data links 132 enable communication between electronic module 134 and controller 106 through connector 116 and socket 130. While module 134 is shown as embedded within connector 116, it is should be understood that module 134 may be positioned anywhere in or on any part of cryoprobe 102, according to convenience of manufacture and/or convenience of use.

Attention is now drawn to FIG. 1B, which presents additional details of electronic module 134. In accordance with an embodiment of the present invention, module 134 comprises read/write memory 136 and/or read-only memory 138, communication interface 140, processor 142 and/or additional electronic components 144 (e.g. sensors, timer, analog/digital converters, etc.). Communication interface 140 provides a data transfer path between module 134 and controller 106.

Referring back to FIG. 1A, controller 106 comprises pressure transducers 146 and servo-controlled valves 148. Pressure transducers 146 measure the pressure of the cryogas in cryogas source 104; and servo-controlled valves 148 regulate, in a manner well known in the art, the flow of cryogas from cryogas source 104 to cryoprobe 102. In some embodiments controller 106 is programmed to regulate the flow of cryogas from cryogas source 104 to cryoprobe 102 in response to information received from module 134. Typically, the cryogas is a cooling gas such as argon. In alternate embodiments, the cryogas is a liquefied gas operable to cool head 112 by evaporation. In other embodiments, source 104 contains a heating gas such as helium for heating portions of cryoprobe 102.

As shown, controller 106 comprises memory 150, processor 152, user interface 154, and communications module 156. User interface 154 includes an input device such as a key board or a touch screen display, and an output device such as display. Various other input and output devices used as user interfaces, as are well known in the art, are also contemplated as alternate embodiments of the instant invention. In accordance with an embodiment of the invention, user interface 154 is used by a surgeon to provide operational and control instructions to controller 106.

In an embodiment of the invention, controller 106 is programmed to calculate and issue commands in response to information received from module 134. In an alternate embodiment, controller 106 is programmed to calculate and issue commands in response to information received from one or more sensors within cryoprobe 102 and/or sensors connected to controller 106 and/or sensors communicating with controller 106. In other embodiments, controller 106 is programmed to calculate and issue commands in response to commands issued by an operator. In yet other embodiments, controller 106 is programmed to calculate and issue commands in response to communications from a remote source (e.g. a network, the Internet, etc.) received through communications module 156.

Controller 106 is operable to read information from memories 136/138 of module 134 and optionally is operable to write information to memory 136 of module 134. Memories 136/138 contain information written during manufacture and/or factory calibration which are accessible to controller 106. Such information includes, but is not limited to, a unique identity code for each cryoprobe 102, cryoprobe type, cryoprobe specifications, test results, etc. Such cryoprobe specific information, readable by controller 106 during power-up (e.g. at the time of initial connection between cryoprobe 102 and controller 106) or at any other time, enables controller 102 determine cryoprobe specific operating parameters in view of a specific treatment plan. For example, electrical properties of electrical heating element 128 such as the change in electrical resistance as a function of temperature is encoded in memories 136/138 of module 134. Such operating characteristics of electrical heating element 128 are then used by controller 106, as described herein below with reference to FIGS. 5, 6 and 7, to regulate the temperature of electrical heating element 128 to specified values by regulating the flow of current through electrical heating element 128 and/or by regulating the voltage applied across electrical heating element 128. Also, for example, since the actual gas throughput of individual cryoprobes under identical cryogas pressure conditions typically varies somewhat, operating characteristics (e.g. cooling capacity) of individual cryoprobes from testing under standard conditions is encoded in memories 136/138 of each individual cryoprobe and subsequently used by controller 106 to determine optimal operating parameters (e.g. length of timed cooling operations). The use of such information provides a more accurately determinable cooling effect than that determinable merely according to theoretical cooling capacities or other characteristics specified only by their intended operating and manufacturing parameters. In alternate embodiments wherein cryoprobe 102 does not include module 134, the surgeon or an operator enters the identification information for cryoprobe 102 via user interface 154, and all necessary cryoprobe specific information is obtained from a configuration file in controller 106.

Controller 106 monitors, records and reports individual and collective cryoprobe usage statistics and limits or otherwise regulates cryoprobe re-use for commercial purposes and/or to enforce safety standards or for other clinical purposes. Testing status, measured operating statistics, activation history, and other cryoprobe specific information is usable to enable/disable use of individual cryoprobes 102. Controller 106 uses communications module 156 for communicating with a remote server, such as a server accessible through the Internet or by other communication means and operated by a manufacturer of cryosurgical system 100 or by a commercial intermediary such as a local supplier of cryosurgical system 100. Such communications is used to report cryoprobe usage patterns, to request and receive authorization for an operation, for inventory management, or for other purposes.

The capabilities mentioned in the preceding paragraph and elsewhere herein constitute a potential advantage of cryoprobe 102 and cryosurgical system 100 over prior art cryoprobes and cryosurgical systems. For example, some cryoprobe manufacturers instruct users to test cryoprobes prior to use, and to avoid excessive re-use, and users may even undertake an obligation to quantitatively limit cryoprobe re-use, yet prior art cryosurgical systems provided no means for verifying such user behavior nor for enforcing these limitations. As shown above, means for such verification and enforcement may be provided by cryosurgical system 100. Cryosurgical system 100 is optionally operable to ensure that only cryoprobes manufactured to be compatible with controller 106 are connected to and used with controller 106 during a surgical procedure.

It is noted that optional electronic components 144 and 158 are installed in module 134 and controller 106, respectively, to provide additional functionality. For example, components 144/150 comprise one or more sensors, timers, analog/digital converters, etc. Such components can be used, for example, as part of a temperature-reporting system wherein an ammeter or a voltmeter or a Wheatstone bridge is used to assess the temperature of electrical heating element 128 as a function of the heating element's electrical characteristics. Other forms of temperature sensors, pressure sensors, flow meters, or other sensors can also be interfaced through module 134 and/or controller 106. In some embodiments, components 144 and 158 comprise radio frequency communications devices or other communications devices enabling wireless communication between two or more of module 134, controller 106, a remote server, a network, the Internet, etc.

Although not explicitly illustrated, cabinet 108 is configured for enabling simultaneous connection of a plurality of cryoprobes 102, and controller 106 is configured for enabling simultaneous control and use of a plurality of cryoprobes 102. For simplicity, FIG. 1A shows only one such connection, viz., for connecting connector 116 on a proximal end of cryoprobe 102 to socket 130 on controller 106. Furthermore, and again for simplicity, FIG. 1A shows only one servo-controlled valve 148 for regulating, in a manner well known in the art, the flow of cryogas from cryogas source 104 to cryoprobe 102. As explained above, controller 106 verifies the identity and type of all cryoprobes 102 connected to controller 106 by communicating with electronic module 134 on each cryoprobe 102 connected to controller 106. Additionally, all cryoprobe-specific information encoded in electronic module 134 is accessible by controller 106. Accordingly, by taking into consideration all probe-specific information, controller 106 modifies the operating parameters of each connected cryoprobe 102. These capabilities enable the user, via controller 106, to tailor parameters such as the temperature for cauterizing the tissue by regulating the current flowing through electrical heating element 128 and/or the voltage applied across electrical heating element 128. Additionally, controller 106 is configured to tailor the cryogen flow times to one or more cryoprobes 102, and thereby facilitate simultaneous use of a plurality of differing types of cryoprobes with the same controller 106 during an entire surgical procedure. Verification that cryoprobes 102 actually connected to controller 106 correspond to those whose connection was planned or intended is an additional feature provided by system 100.

An additional optional use of the cryosurgical system described herein above is to facilitate the use of cryoprobes of differing capacities simultaneously or sequentially with a common controller 106. Since each cryoprobe 102 is configured to supply self-descriptive information, controller 106 can be programmed to adapt its operational parameters to each cryoprobe individually, thus enabling a mixture of a plurality of cryoprobes with differing cooling and/or heating capacities or other differing operational characteristics and yet easily cause each cryoprobe to conform to a pre-determined common surgical plan (e.g. a planned ice-ball shape and size). As such, it is also possible to determine whether the characteristics of cryoprobes 102 actually connected to controller 106 correspond to cryoprobe characteristics called for in a surgical plan, thereby assuring that correctly characterized cryoprobes are inserted and used.

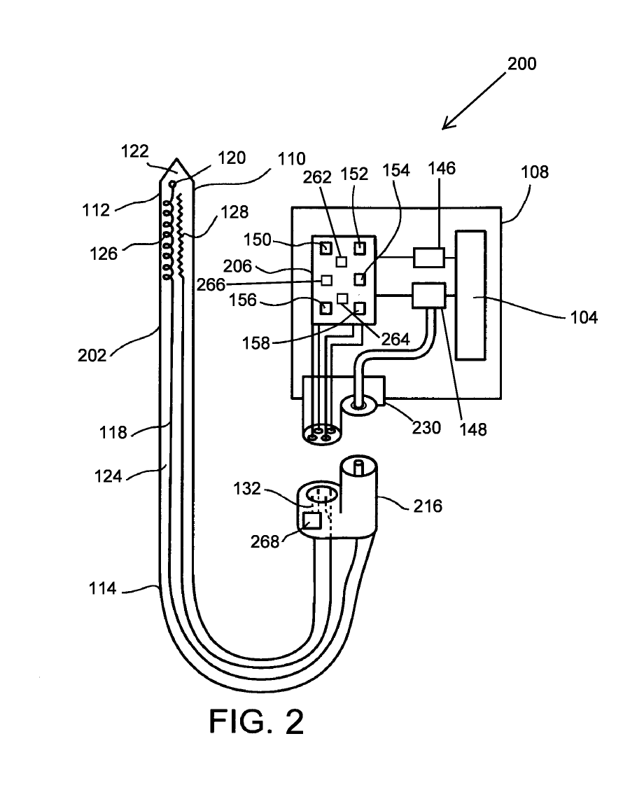

An alternate embodiment of cryosurgical system 100 described herein above with reference to FIGS. 1A and 1B is illustrated in FIG. 2 as cryosurgical system 200, wherein like elements are represented by like numerals. Accordingly, and in the interest of brevity, the following description in reference to FIG. 2 focuses only on those elements of cryosurgical system 200 that are different from the embodiments of cryosurgical system 100.

As with cryosurgical system 100, cryosurgical system 200 comprises cryoprobe 202, cryogas source 104 for supplying a cryogas to cryoprobe 202, and controller 206 for controlling delivery of cryogas from cryogas source 104 to cryoprobe 202.

In an embodiment of the invention, controller 206 includes data source 262 for obtaining cryoprobe specific information from a database within controller 206. In alternate embodiments, data source 262 is an interface for receiving cryoprobe specific information over a network or over the Internet or via wireless communication.

Controller 206 further comprises query module 264 for transmitting one or more query signals when one or more cryoprobes 202 are first connected to controller 206 or at any other time. In an embodiment of the invention, query module 264 functions to formulate, based on information received from data source 262, a query signal for transmission to cryoprobe 202. In an alternate embodiment, query module 264 transmits a series of query signals to cryoprobe 202 based on information known to controller 206 about one or more cryoprobes 202.

Controller 206 further comprises identifier module 266, for receiving from cryoprobe 202 a response to a query signal transmitted by query module 264, and for analyzing that response signal to determine if it is possible, based on that signal, to establish a unique cryoprobe-specific identity code for cryoprobe 202.

In an embodiment of the invention, connector 216 on a proximal portion of cryoprobe 202 includes response module 268 in the form of an electronic circuit. In an alternate embodiment, response module 268 is an embedded radio-frequency (RF) tag. Response module 268 is operable to recognize when a received query signal, transmitted by query module 264, possesses a predetermined characteristic, and to emit a characteristic response, which can be an encoded signal or a simple signal, when a query signal having said predetermined characteristic is recognized. In the simple embodiment mentioned above, wherein query signals are unique cryoprobe-specific identity codes, response module 268 simply tests an incoming signal to determine whether the incoming signal is recognized as its own unique cryoprobe-specific identity code. If it is, response module 268 transmits a “yes” response, whereby the cryoprobe is identified and the query process terminates. If it is not, response module 268 transmits a “no” response or does not transmit anything. In the event of a “no” or no response, query module 264 then transmits other queries based on information about other cryoprobes in its data list (obtained from data source 262 or any other source), cycling through its list of known cryoprobes until a match is found. In an alternate embodiment, query module 264 transmits a real-time date and asks for a response from cryoprobes whose expiration date is prior to, or alternatively later than, the transmitted real-time date. Response module 268 comprising a memory containing an expiration date can recognize a query signal encoding a real-time date, and appropriately transmit a “yes” or a “no” response.

At that point, controller 206 knows which of the cryoprobes 202 known to it is attached at the position to which the queries are sent. From then on, the various procedures and methods outlined above with respect to cryosurgical system 100 are undertaken. Information read from data source 262 and now associated with specific cryoprobes 202 connected to controller 206 can include information characterizing a usage history of such cryoprobes, data derived from an operational test of the cryoprobes, a type designation for the cryoprobes, a descriptive characterization of the cryoprobes, electrical properties of electrical heating element 128 such as the change in electrical resistance as a function of temperature, etc.

Although not explicitly illustrated, cabinet 108 is configured for enabling simultaneous connection of a plurality of cryoprobes 202, and controller 206 is configured for enabling simultaneous control and use of a plurality of cryoprobes 202. For simplicity, FIG. 2 shows only one such connection, viz., for connecting connector 216 on a proximal end of cryoprobe 202 to socket 230 on controller 206. Furthermore, and again for simplicity, FIG. 2 shows only one servo-controlled valve 148 for regulating, in a manner well known in the art, the flow of cryogas from cryogas source 104 to cryoprobe 202. As explained above, probe-specific information obtained from data source 262 is used by controller 206 to verify the identity and type of all cryoprobes 202 connected to controller 206 and to modify the operating parameters of each connected cryoprobe 202. These capabilities enable controller 206 to tailor parameters such as the temperature for cauterizing the tissue by regulating the current flowing through electrical heating element 128 and/or the voltage applied across electrical heating element 128. Additionally, controller 206 is configured to tailor the cryogen flow times to one or more cryoprobes 202, and thereby facilitate simultaneous use of a plurality of differing types of cryoprobes with the same controller 206 during an entire surgical procedure. Verification that cryoprobes 202 actually connected to controller 206 correspond to those whose connection was planned or intended is an additional feature provided by system 200.

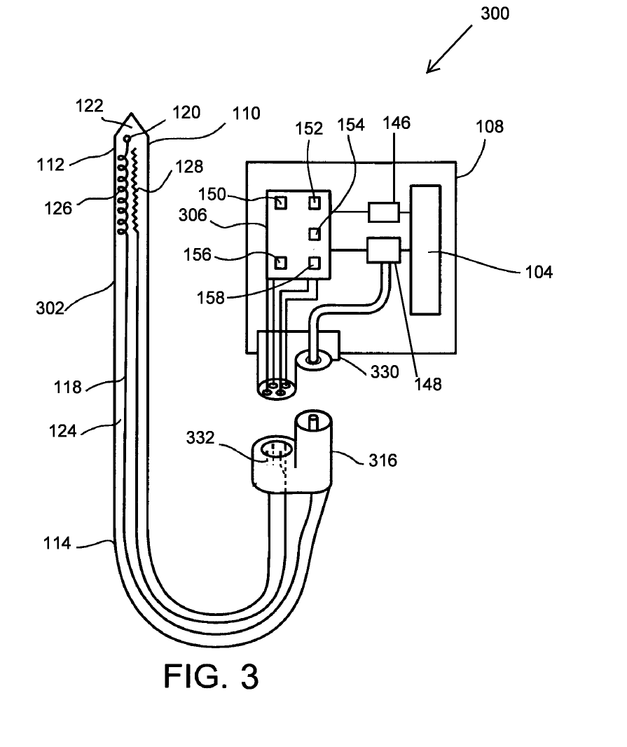

Another embodiment of cryosurgical systems 100 and 200 described herein above with reference to FIGS. 1A and 2 is illustrated in FIG. 3 as cryosurgical system 300, wherein like elements are represented by like numerals. Accordingly, and in the interest of brevity, the following description in reference to FIG. 3 focuses only on those elements of cryosurgical system 300 that are different from the embodiments of cryosurgical systems 100 and 200.

FIG. 3 illustrates cryosurgical system 300 comprising cryoprobe 302, cryogas source 104 for supplying a cryogas to cryoprobe 302, and controller 306 for controlling delivery of cryogas from cryogas source 104 to cryoprobe 302. Connector 316 on a proximal portion of cryoprobe 302 is used for connecting cryoprobe 302 to socket 330 on cabinet 108 for providing gas connection to cryogas source 104 and electrical connection to controller 306. In this embodiment, connector 316 includes power link 332 but no data link. Power link 332 supplies electrical power to cryoprobe 302, such as to electrical heating element 128. As shown, connector 316 does not include a communications link or a response module such as power and data links 132 in connector 116 or response module 268 in connector 216.

Furthermore, power and data links 132 enable communication between electronic module 134 and controller 106 through connector 116 and socket 130. While module 134 is shown as embedded within connector 116, it is should be understood that module 134 may be positioned anywhere in or on any part of cryoprobe 102, according to convenience of manufacture and/or convenience of use.

Additionally, in an embodiment of cryosurgical system 300, controller 306 does not include any other automated means for obtaining any cryoprobe identity codes and/or any other cryoprobe-specific information. For instance, controller 306 does not include a data source such as data source 262 in an embodiment of controller 206.

As described in the foregoing with reference to controllers 106 and 206, controller 306 in an embodiment of cryosurgical system 300 is programmed to calculate and issue commands and, in general, to operate cryosurgical system 300 in response to one or more of commands issued by an operator or in response to information received from one or more sensors connected to and/or communicating with controller 306 or in response to communications from a remote source (e.g. a network, the Internet, etc.). Accordingly, user interface 154 is used for providing all cryoprobe-specific information to controller 306 for every cryoprobe 302 connected to controller 306. In accordance with an embodiment of the invention, cryoprobe-specific information is provided to controller 306 in a sequential manner, i.e., as each cryoprobe 302 is placed within a tissue and connected to controller 306 via socket 330, the surgeon configures controller 306 to recognize the newly connected cryoprobe 302 and thereafter enters the cryoprobe-specific information in an associative manner.

User interface 154 includes an input device such as a key board or a touch screen display, and an output device such as display. Various other input and output devices used as user interfaces, as are well known in the art, are also contemplated as alternate embodiments of the instant invention. In accordance with an embodiment of the invention, user interface 154 is used by a surgeon to provide operational and control instructions to controller 306.

As described in the foregoing with reference to cryosurgical systems 100 and 200, the information provided to controller 306 through user interface 154 typically includes all or a subset of the information (e.g., the unique identity code of each cryoprobe 302, cryoprobe type, cryoprobe specifications, test results, actual gas throughput, electrical properties of electrical heating element 128 such as the change in electrical resistance as a function of temperature, etc.) provided to controller 106 via electronic module 134 or provided to controller 206 via data source 262. Such information enables controller 306 to tailor parameters such as the temperature for cauterizing the tissue by regulating the current flowing through electrical heating element 128 and/or the voltage applied across electrical heating element 128. Additionally, controller 306 is configured to tailor the cryogen flow times to one or more cryoprobes 302, and thereby facilitate simultaneous use of a plurality of differing types of cryoprobes with the same controller 306 during an entire surgical procedure.

Although not explicitly illustrated, cabinet 108 is configured for enabling simultaneous connection of a plurality of cryoprobes 302, and controller 306 is configured for enabling simultaneous control and use of a plurality of cryoprobes 302. For simplicity, FIG. 3 shows only one such connection, viz., for connecting connector 316 on a proximal end of cryoprobe 302 to socket 330 on controller 306. Furthermore, and again for simplicity, FIG. 3 shows only one servo-controlled valve 148 for regulating, in a manner well known in the art, the flow of cryogas from cryogas source 104 to cryoprobe 302.

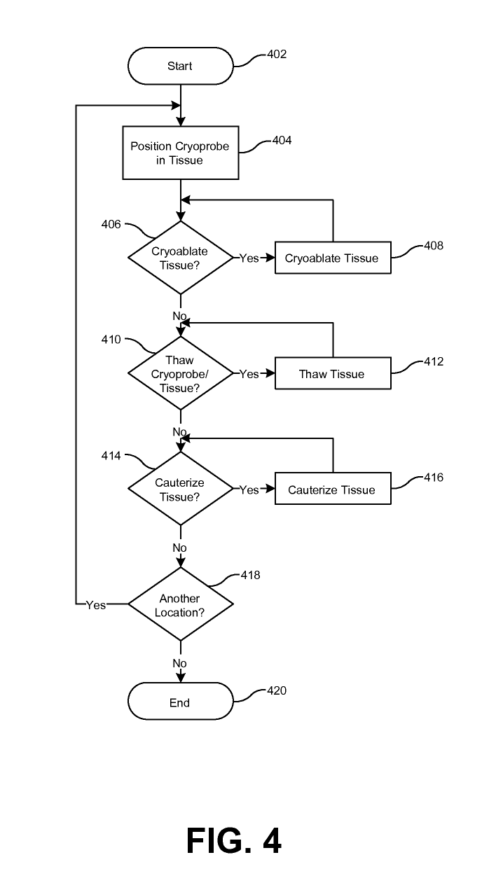

In accordance with an embodiment of the invention, FIG. 4 is a flowchart of an exemplary method for using a cryoprobe during a cryosurgical procedure for performing one or more of cryoablating a tissue, thawing a tissue to dis-engage the cryoprobe frozen to the tissue, and cauterizing the tissue. The method, starting at block 402, may be implemented as a stand-alone program running on controller 106/206/306 or it may be a subroutine or a sub-program executed under the control of one or more other programs running on controller 106/206/306. At block 404, the surgeon positions the cryoprobe at the location where the tissue will be cryoablated, thawed and/or cauterized. In some instances, the surgical plan including the operation to be performed on the tissue, i.e., whether the tissue will be cryoablated, thawed and/or cauterized, and the sequence in which the operation will be or should be performed may have been previously entered or programmed into controller 106/206/306 by the surgeon. In other instances, the surgeon may change the surgical plan during a surgical procedure and either specify a new surgical plan into controller 106/206/306 or the surgeon may override a previously specified surgical plan.

Accordingly, at block 406, controller 106/206/306 checks whether or not the tissue needs to be cryoablated based on either a pre-specified surgical plan or as directed by the surgeon. If the tissue needs to be cryoablated, then controller 106/206/306 issues the appropriate commands at block 408 to cryoablate the tissue in accordance with procedures as are well known in the art. The one or more command issued at block 408 includes the introduction of the cooling gas from gas source 104 and its subsequent expansion across Joule-Thomson orifice 120. Upon completion, control is transferred back to block 406 to determine if the surgical plan calls for additional cryoablation. This process continues until cryoablation is completed or canceled and no further cryoablation is required at the current tissue location.

Subsequently, control passes to block 410 whereat controller 106/206/306 checks whether or not the thaw cycle should be initiated for un-freezing and dis-engaging cryoprobe 102/202/302 from the cryoablated tissue. The thaw cycle may be initiated based on either a pre-specified surgical plan or as directed by the surgeon. If the tissue needs to be thawed, then controller 106/206/306 issues the appropriate commands at block 412 to heat treatment head 112 to a temperature in the range of 20° C. to 50° C. for thawing the tissue in accordance with procedures as are well known in the art. The one or more command issued at block 412 includes the introduction of the heating gas from gas source 104 and its subsequent expansion across Joule-Thomson orifice 120. Upon completion, control is transferred back to block 410 to determine if the surgical plan calls for additional thawing. This process continues until thawing is completed or canceled and no further thawing is required at the current tissue location.

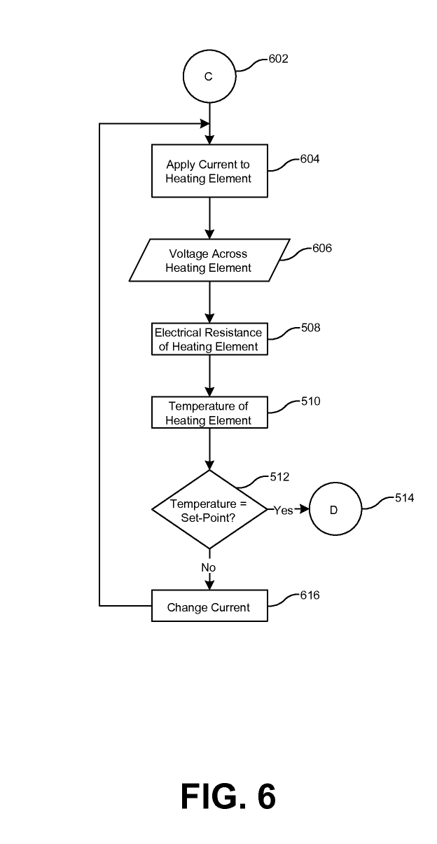

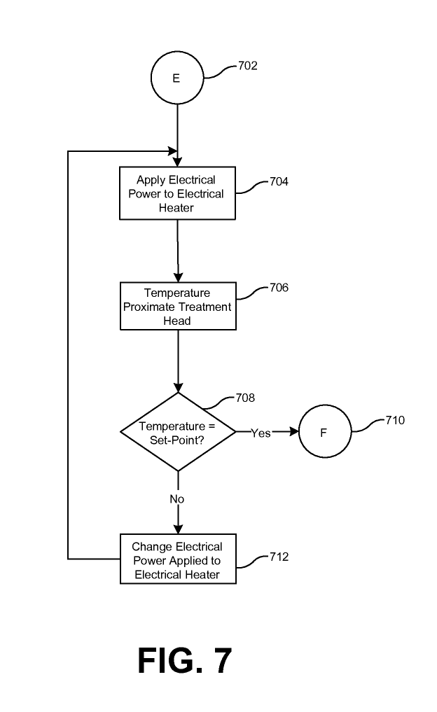

Next, control passes to block 414 whereat controller 106/206/306 checks whether or not the cauterization cycle should be initiated for cauterizing the tissue. The cauterization cycle may be initiated based on either a pre-specified surgical plan or as directed by the surgeon. If the tissue needs to be cauterized, then controller 106/206/306 issues the appropriate commands at block 416 to heat treatment head 112 to a specified temperature sufficiently high to cauterize the tissue (for example between 85° C. and 120° C.). In accordance with an embodiment of the invention, the commands issued by controller 106/206/306 at block 416 for heating treatment head 112 are described herein below with reference to FIG. 5. In an alternate embodiment of the invention, the commands issued by controller 106/206/306 at block 416 for heating treatment head 112 are described herein below with reference to FIG. 6. In another embodiment of the invention, the commands issued by controller 106/206/306 at block 416 for heating treatment head 112 are described herein below with reference to FIG. 7. Upon completion, control is transferred back to block 418 to determine if the surgical plan calls for additional cauterization. This process continues until cauterization is completed or canceled and no further cauterization is required at the current tissue location.

Next, at block 418, controller 106/206/306 checks whether the pre-specified surgical plan calls for additional cryosurgery, i.e., whether cryoablation, thawing and/or cauterization. The additional cryosurgery may be performed at the same tissue location or at another location. If additional cryosurgery is required, control is transferred to block 404. If additional cryosurgery is not required, then the procedure terminates at block 420.

In accordance with an embodiment of the invention, FIG. 5 is a flowchart of a method for heating treatment head 112 and maintaining the temperature of electrical heating element 128 within a predetermined range by regulating the power supplied to electrical heating element 128. In this embodiment, the power supplied to electrical heating element 128 is regulated by regulating the electrical voltage applied across electrical heating element 128. The method illustrated in FIG. 5, which is an alternative to the methods described herein below with reference to FIGS. 6 and 7, is associated with block 416 of FIG. 4 for cauterizing the tissue. Starting at block 502, controller 106/206/306 applies a pre-determined electrical voltage across electrical heating element 128 at block 504. As is well known in the art, an application of an electrical voltage across an electrical conductor, such as electrical heating element 128, will induce a flow of electrical current through the electrical conductor and also heat the electrical conductor causing its temperature to increase. The resultant electrical current flowing through electrical heating element 128 is measured by controller 106/206/306 at block 506. Next, in accordance with algorithms well known in the art, at block 508 controller 106/206/306 computes the electrical resistance of electrical heating element 128. As described in the foregoing, electrical heating element 128, in some embodiments of the invention, exhibits a negative temperature coefficient characteristic in that the electrical resistance of electrical heating element 128 decreases as the temperature of electrical heating element 128 increases. In alternate embodiments of the invention, electrical heating element 128 exhibits a positive temperature coefficient characteristic in that the electrical resistance of electrical heating element 128 increases as the temperature of electrical heating element 128 increases. As such, the relationship between the electrical resistance and the temperature of electrical heating element 128 is usually known or can be easily determined. Accordingly, at block 510, controller 106/206/306 computes the temperature of electrical heating element 128 using the electrical resistance computed at block 508 and the known relationship between the electrical resistance and the temperature of electrical heating element 128. Then, at block 512, controller 106/206/306 checks whether the temperature of electrical heating element 128 is within a range of a pre-defined set point temperature which is sufficiently for cauterizing the tissue. If the temperature of electrical heating element 128 is within the range of a pre-defined set point temperature, then the tissue is cauterized and the method exits at block 514 and thereafter continues at block 416. However, if the temperature of electrical heating element 128 is outside the range of a pre-defined set point temperature, then the temperature of electrical heating element 128 is either too low for cauterizing the tissue or is too high, and must be adjusted. Accordingly, at block 516, controller 106/206/306 adjusts the electrical voltage applied across electrical heating element 128, and the method continues at block 504. As is well known in the art, a change in the electrical voltage applied across electrical heating element 128 will also change the electrical current flowing through electrical heating element 128. Accordingly, the electrical power supplied to electrical heating element 128 will also change.

FIG. 6 is a flowchart of another embodiment of the invention of a method for heating treatment head 112 and maintaining the temperature of electrical heating element 128 within a predetermined range by regulating the power supplied to electrical heating element 128. In this embodiment, the power supplied to electrical heating element 128 is regulated by regulating the electrical current supplied to electrical heating element 128. The method illustrated in FIG. 6, which is an alternative to the methods described with reference to FIGS. 5 and 7, is also associated with block 416 of FIG. 4 for cauterizing the tissue. Steps that are identical between the methods shown in FIGS. 5 and 6 are identified by like numerals. Starting at block 602, controller 106/206/306 supplies a pre-determined electrical current to electrical heating element 128 at block 604. As is well known in the art, supplying electrical current to an electrical conductor, such as electrical heating element 128, will heat the electrical conductor causing its temperature to increase and will also establish an electrical voltage across the electrical conductor. The electrical voltage across electrical heating element 128 is measured by controller 106/206/306 at block 606. Next, controller 106/206/306 executes blocks 508 through 514, inclusive, as described herein above with reference to FIG. 5, which steps are not repeated here in the interest of brevity. If, at block 512, controller 106/206/306 determines that the temperature of electrical heating element 128 is outside the range of a pre-defined set point temperature, then, at block 616, controller 106/206/306 adjusts the electrical current being supplied to electrical heating element 128, and the method continues at block 604. As is well known in the art, a change in the electrical current supplied to electrical heating element 128 will also change the electrical voltage across electrical heating element 128. Accordingly, the electrical power supplied to electrical heating element 128 will also change.

As described in the foregoing, embodiments of cryosurgical system 100/200/300 are configured to include a temperature sensor in communication with controller 106/206/306. As described herein above with reference to FIGS. 5 and 6, electrical heating element 128 also functions as a temperature sensor in some embodiments of the invention. In other embodiments, a separate temperature sensor and/or a temperature sensor in addition to electrical heating element 128 is used for measuring the temperature in the vicinity of electrical heating element 128. As described herein below with reference to FIG. 7, temperature measurements from one or more temperature sensors located proximate electrical heating element 128 can also be used for regulating the electrical power applied to electrical heating element 128 for maintaining the temperatures within the range of a pre-defined set point temperature necessary for cauterizing the tissue.

FIG. 7 is a flowchart of an alternative embodiment of a method for heating treatment head 112 and maintaining the temperature in the vicinity of electrical heating element 128 within a predetermined range by regulating the power supplied to electrical heating element 128. The method illustrated in FIG. 7, which is an alternative to the methods described herein above with reference to FIGS. 5 and 6, is also associated with block 416 of FIG. 4 for cauterizing the tissue. Starting at block 702, and using methods well known in the art, controller 106/206/306 supplies electrical power to electrical heating element 128 at block 704. Supplying electrical power to an electrical conductor, such as electrical heating element 128, will induce a flow of electrical current through the electrical conductor and also heat the electrical conductor causing its temperature to increase. Next, at block 708, the temperature proximate treatment head 112 is measured by controller 106/206/306 using the one or more temperature sensors connected thereto. Then, at block 708, controller 106/206/306 checks whether the measured temperature is within a range of a pre-defined set point temperature which is sufficiently for cauterizing the tissue. If the measured temperature is within the range of a pre-defined set point temperature, then the tissue is cauterized and the method exits at block 710 and thereafter continues at block 416. However, if the measured temperature is outside the range of a pre-defined set point temperature, then the electrical power supplied to electrical heating element 128 must be adjusted. Accordingly, at block 712, controller 106/206/306 adjusts the electrical power supplied to electrical heating element 128, and the method continues at block 704.

Various modifications and additions may be made to the exemplary embodiments presented hereinabove without departing from the spirit, scope and intent of the present invention. For example, while the disclosed embodiments refer to particular features, the scope of the instant invention is considered to also include embodiments having various combinations of features different from and/or in addition to those described hereinabove. Accordingly, the present invention embraces all such alternatives, modifications, and variations as within the spirit, scope and intent of the appended claims, including all equivalents thereof.

We claim:

1. A method of treating a tissue, comprising
positioning a cryoprobe at a location in the tissue being treated; and
operating the cryoprobe to
cryoablate the tissue;
thaw the cryoablated tissue; and
thermally cauterize the tissue.

2. The method of claim 1, wherein operating the cryoprobe to cryoablate the tissue comprises regulating a flow of a cooling gas to the cryoprobe.

3. The method of claim 1, wherein operating the cryoprobe to
thaw the cryoablated tissue comprises heating the cryoprobe to a temperature sufficient to thaw the cryoablated tissue; and
thermally cauterize the tissue comprises heating the cryoprobe to a temperature sufficient to cauterize the tissue.

4. The method of claim 3, comprising maintaining the temperature in the range of 85° C. to 120° C. for thermally cauterizing the tissue.

5. The method of claim 3, comprising maintaining the temperature in the range of 20° C. to 50° C. for thawing the cryoablated tissue.

6. The method of claim 5, further comprising regulating a flow of a heating gas to the cryoprobe.

7. The method of claim 3, wherein heating the cryoprobe comprises supplying electrical power to an electrical heating element located proximate a tip of the cryoprobe.

8. The method of claim 7, comprising
applying a predetermined electrical voltage across the electrical heating element;
measuring an electrical current flowing through the electrical heating element;
computing an electrical resistance of the electrical heating element;
computing a temperature of the electrical heating element as represented by the electrical resistance of the electrical heating element; and
maintaining the temperature of the electrical heating element within a predetermined range by regulating the electrical voltage applied across the electrical heating element.

9. The method of claim 7, comprising
supplying a predetermined electrical current to the electrical heating element;
measuring an electrical voltage across the electrical heating element;
computing an electrical resistance of the electrical heating element;
computing a temperature of the electrical heating element as represented by the electrical resistance; and
maintaining the temperature of the electrical heating element within a predetermined range by regulating the electrical current supplied to the electrical heating element.

10. The method of claim 7, comprising
measuring a temperature proximate the electrical heating element; and
maintaining the temperature within a predetermined range by regulating the electrical power supplied to the electrical heating element.

11. The method of claim 7, further comprising regulating said electrical power supplied to said electrical heating element by regulating an electrical voltage applied across said electrical heating element.

12. The method of claim 7, further comprising regulating said electrical power supplied to said electrical heating element by regulating a flow of electrical current flowing through said electrical heating element.

13. A means for treating a tissue, comprising
means for positioning a cryoprobe at a location in the tissue being treated; and
means for operating the cryoprobe to
cryoablate the tissue;
thaw the cryoablated tissue; and
thermally cauterize the tissue.

14. The means for claim 13, wherein the means for operating the cryoprobe to cryoablate the tissue comprises means for regulating a flow of a cooling gas to the cryoprobe.

15. The means for claim 13, wherein the means for operating the cryoprobe to
thaw the cryoablated tissue comprises means for heating the cryoprobe to a temperature sufficient to thaw the cryoablated tissue; and
thermally cauterize the tissue comprises means for heating the cryoprobe to a temperature sufficient to cauterize the tissue.

16. The means for claim 15, comprising means for maintaining the temperature in the range of 85° C. to 120° C. for thermally cauterizing the tissue.

17. The means for claim 15, comprising means for maintaining the temperature in the range of 20° C. to 50° C. for thawing the cryoablated tissue.

18. The means for claim 17, further comprising means for regulating a flow of a heating gas to the cryoprobe.

19. The means for claim 15, wherein the means for heating the cryoprobe comprises means for supplying electrical power to an electrical heating element located proximate a tip of the cryoprobe.

20. The means for claim 19, comprising
means for applying a predetermined electrical voltage across the electrical heating element;
means for measuring an electrical current flowing through the electrical heating element;
means for computing an electrical resistance of the electrical heating element;
means for computing a temperature of the electrical heating element as represented by the electrical resistance; and
means for maintaining the temperature of the electrical heating element within a predetermined range by regulating the electrical voltage applied across the electrical heating element.

21. The means for claim 19, comprising
means for supplying a predetermined electrical current to the electrical heating element;
means for measuring an electrical voltage across the electrical heating element;
means for computing an electrical resistance of the electrical heating element;
means for computing a temperature of the electrical heating element as represented by the electrical resistance; and
means for maintaining the temperature of the electrical heating element within a predetermined range by regulating the electrical current supplied to the electrical heating element.

22. The means for claim 19, comprising
means for measuring a temperature proximate the electrical heating element; and
means for maintaining the temperature within a predetermined range by regulating the electrical power supplied to the electrical heating element.

23. The means for claim 19, comprising means for regulating said electrical power supplied to said electrical heating element using means for regulating an electrical voltage applied across said electrical heating element.

24. The means for claim 19, comprising means for regulating said electrical power supplied to said electrical heating element using means for regulating a flow of electrical current flowing through said electrical heating element.

25. A cryosurgical system, comprising
a cryoprobe including an electrical heating element at a distal end thereof, said cryoprobe configured for
cryoablating a tissue;
thawing the cryoablated tissue; and
cauterizing the tissue; and
a controller configured for maintaining a temperature proximate the distal end within a predetermined range.

26. The cryosurgical system of claim 25, further comprising a cryogas source, wherein the controller is configured for regulating a flow of the cryogas from the cryogas source to the cryoprobe.

27. The cryosurgical system of claim 26, wherein the cryogas is a cooling gas.

28. The cryosurgical system of claim 26, wherein the cryogas is a heating gas.

29. The cryosurgical system of claim 26, wherein the controller is configured for supplying electrical power to the electrical heating element.

30. The cryosurgical system of claim 29, wherein the controller
applies an electrical voltage across the electrical heating element;
receives a signal indicative of an electrical current flowing through the electrical heating element;
computes an electrical resistance of the electrical heating element;
computes a temperature of the electrical heating element as represented by the electrical resistance of the electrical heating element; and
maintains the temperature of the electrical heating element within a predetermined range by regulating the electrical voltage applied across the electrical heating element.

31. The cryosurgical system of claim 29, wherein the controller supplies an electrical current to the electrical heating element;
receives a signal indicative of an electrical voltage across the electrical heating element;
computes an electrical resistance of the electrical heating element;
computes a temperature of the electrical heating element as represented by the electrical resistance of the electrical heating element; and
maintains the temperature of the electrical heating element within a predetermined range by regulating the electrical current supplied to the electrical heating element.

32. The cryosurgical system of claim 29, wherein the controller
receives a signal indicative of the temperature proximate the distal end; and
maintains the temperature within a predetermined range by regulating the electrical power supplied to the electrical heating element.

33. The cryosurgical system of claim 29, wherein the controller regulates said electrical power supplied to said electrical heating element by regulating an electrical voltage applied across said electrical heating element.

34. The cryosurgical system of claim 29, wherein the controller regulates said electrical power supplied to said electrical heating element by regulating a flow of electrical current flowing through said electrical heating element.

35. The cryosurgical system of claim 26, wherein the controller maintains the temperature in the range of 85° C. to 120° C. for thermally cauterizing the tissue.

36. The cryosurgical system of claim 26, wherein the controller maintains the temperature in the range of 20° C. to 50° C. for thawing the cryoablated tissue.
